(19) United States
(12) Patent Application Publication
(10) Pub. No.: US 2017/0007279 A1
(43) Pub. Date: Jan. 12, 2017

(54) Variable Flexibility Snare

(71) Applicant: Virender K. Sharma, Paradise Valley, AZ (US)

(72) Inventor: Virender K. Sharma, Paradise Valley, AZ (US)

(21) Appl. No.: 15/155,030

(22) Filed: May 15, 2016

Related U.S. Application Data
(60) Provisional application No. 62/162,633, filed on May 15, 2015.

(51) Int. Cl. A61B 17/221; A61B 18/14

(52) U.S. Cl. CPC A61B 17/221; A61B 18/14; A61B 2017/2212; A61B 2018/00595

(57) ABSTRACT

Variable size snare devices include devices which can be reliably and predictably opened to the required size, can be reliably and predictably opened to the required shape, and can provide visual cues indicative of the size of the deployed snare loop. The snare devices also include variable stiffness levels wherein the devices comprise shape memory alloys such that the stiffness of the snare loop can be controlled for desirable effect.

### CROSS-REFERENCE

The present application relies on U.S. Provisional Patent Application No. 62/162,633, entitled “Variable Size Snare” and filed on May 15, 2015, for priority, which is herein incorporated by reference in its entirety.

### FIELD

The present specification relates to snare devices used in medical procedures. More particularly, the present specification relates to a snare device having a variably flexible snare.

### BACKGROUND

A snare is a surgical instrument with a wire loop controlled by a mechanism in the handle, used to remove growths, such as tumors or polyps in medical procedures. Snares devices have found great use in conducting medical procedures in a minimally invasive manner. The snare device can be passed through the working channel of an endoscope such that the wire loop of the snare device encircles the target tissue. The wire loop is then drawn into a sheath coupled with the wire loop to sever the target tissue. In certain cases, electrocautery can also be performed by applying a current to the wire loop to assist in removing the tissue.

In medical procedures, tumors or polyps might be of various sizes and, in cases when the size of the snare loop is not aligned with the size of the polyp, the practitioner is required to completely withdraw the snare device and insert an alternate snare device, which makes the entire process very tedious. Further, the healthcare facility has to keep a stock of multiple snare devices having different sizes. Therefore, it is important to have snare devices in which the size of the snare or the wire loop can be modified as per the requirement. A variable size snare loop would simplify medical procedures by allowing a practitioner to resize the snare loop without completely removing the snare device and inserting an alternate snare device. For example, when utilizing a variable loop snare, a practitioner may simply resize a variable snare loop during the course of the procedure, obviating the need to withdraw the snare loop and associated shaft and replace it with a snare loop of a different size. However, in current devices, it is difficult to control the size of the snare loop. There exists no good method of measuring the loop size and, adjusting the loop size distorts the shape of the loop such that it becomes long or elongated in shape while it is preferred to have a loop which is round in shape.

Also it is desirable to have a snare that has variable flexibility. Soft snare loops conform to angulation in the anatomy however, when the snare needs to be tightened, it is desirable for the loop to stiffen so that is can compress around the lesion and help grasp it better.

Therefore, it is desirable to have a variable size snare device in which the snare can be reliably and predictably opened to the required size, can be reliably and predictably opened to the required shape, and which also provides visual cues indicating the level of opening of the snare. Further, it is also desirable to have a variable stiffness snare in which the stiffness level of the snare loop could be controlled for desirable effect.

### SUMMARY

The present specification describes a variable size snare device comprising: a snare sheath comprising a proximal end and a distal end; a snare wire comprising a proximal end and a distal end and disposed within said snare sheath; a handle coupled to the proximal end of the snare wire; a snare loop coupled to the distal end of the snare wire, said snare loop comprising: a first section having a proximal end and a distal end, the proximal end coupled to the distal end of the snare wire; a second section having a proximal end and a distal end, the proximal end coupled to the distal end of the snare wire; and a third section coupled to and between the distal end of first section and the distal end of second section, wherein each of said first section and second section comprises a plurality of angulations such that the loop sections between such angulations in the first section are aligned with the corresponding loop sections between such angulations in the second section.

Optionally, the number of angulations and the degree of angulations in the first section is similar to the corresponding angulations in the second section. Optionally, each of said first section and said second section comprise three angulations.

Optionally, the length of loop section between any two angulations in the first section is equal to the length of loop section between corresponding set of angulations in the second section. Optionally, the size of the snare loop is increased or decreased by extending or pulling portions of snare loop inside the sheath without substantially distorting the original shape of the snare loop.

Optionally, the presence of said angulations ensures that the general shape of the snare loop is not distorted as portions of snare loop are drawn into the snare sheath. Optionally, the size of the snare loop is increased or decreased in discrete steps without substantially distorting the original shape of the snare loop by extending or withdrawing the entire loop sections between any two angulations in the snare sheath.

Optionally, said third section is substantially circular in shape.

Optionally, the loop sections between any two angulations are colored in different colors to provide a visual cue on a monitor indicative of the size of deployment of the snare loop to an operator. Optionally, the angulations are marked in different colors to provide a visual cue indicative of the size of deployment of the snare loop to an operator.

Optionally, said snare wire disposed inside said snare sheath comprises two wires such that the proximal ends of the wires are coupled to the handle and the distal ends of the wires are coupled to the snare loop and the position and size of the snare loop is controlled through said wires.

Optionally, said snare loop is manufactured from a shape memory alloy and has different stiffness levels across certain range of temperature. Optionally, said handle comprises an infusion port to infuse a suitable fluid into the snare sheath to modify the temperature and stiffness level of the snare loop. Optionally, said shape memory alloy comprises Nitinol. Optionally, said handle comprises an infusion port to infuse a saline solution having a thermal temperature less than the austenite finish (Af) temperature of the Nitinol. Optionally, said snare device further comprises an electrosurgical connector to facilitate electrocautery.

The present specification also describes a variable size snare device comprising: a snare sheath; and a snare wire positioned within said sheath and attached to a handle at one end and having a snare loop at the other end, wherein the snare loop includes a plurality of angulations which allow the snare loop to maintain its shape as it is withdrawn into said snare sheath.

The present specification also describes a snare device with variable stiffness level comprising: a snare sheath; a handle coupled to the proximal end of the snare sheath and comprising an infusion port to infuse fluid inside said sheath; and a snare wire positioned within said sheath and attached to a handle at one end and comprising a snare loop at the other end, wherein the stiffness level of snare loop inside the snare sheath is modified as compared to the stiffness level of the snare loop by bringing it in contact with a fluid infused through said infusion port.

Optionally, the temperature of said fluid is set such that it affects the stiffness level of said snare loop. Optionally, said snare loop is pulled in and out of the distal end of said sheath to change the size and overall stiffness level of deployed snare loop.

The aforementioned and other embodiments of the present invention shall be described in greater depth in the drawings and detailed description provided below.

### BRIEF DESCRIPTION OF THE DRAWINGS

These and other features and advantages of the present invention will be further appreciated, as they become better understood by reference to the detailed description when considered in connection with the accompanying drawings, wherein:

FIG. 1 is an illustration of a typical snare device, depicting a handle, snare loop, and snare sheath;

FIG. 2 is an illustration of a snare device deployed in a working channel of an endoscope, depicting a snare loop encircling a target tissue;

FIG. 3 is an illustration of a snare device handle;

FIG. 4 is another illustration of a snare device, depicting a snare handle, snare loop, and snare sheath;

FIG. 5 is an illustration of multiple snare loops configured such that their loop size can be modified;

FIG. 6 is an illustration of a variety of snare loops having different shapes;

FIG. 7A illustrates a variable size snare device in accordance with an embodiment of the present specification;

FIG. 7B illustrates how the size of the snare loop of FIG. 7A is modified without distorting the shape of the snare loop, in accordance with an embodiment of the present specification;

FIG. 7C is another illustration of how the size of the snare loop of FIG. 7A is modified without distorting the shape of the snare loop, in accordance with an embodiment of the present specification;

FIG. 7D illustrates a snare loop completely drawn into a snare sheath in accordance with an embodiment of the present specification;

FIG. 8A illustrates a variable size snare device, having a snare loop of a first shape, in accordance with another embodiment of the present specification;

FIG. 8B illustrates a variable size snare device, having a snare loop of a second shape, in accordance with yet another embodiment of the present specification;

FIG. 8C illustrates a first stage of constriction of the size of the snare loop of FIG. 8B without distorting the general shape of the snare loop, in accordance with an embodiment of the present specification;

FIG. 8D illustrates a second stage of constriction of the size of the snare loop of FIG. 8B without distorting the general shape of the snare loop, in accordance with an embodiment of the present specification;

FIG. 9A illustrates a snare device having different colors or markers on the snare loop to indicate the size of the loop, in accordance with an embodiment of the present specification;

FIG. 9B illustrates another snare device having colors or markers on the snare loop to indicate the size of the loop, in accordance with an embodiment of the present specification;

FIG. 10 is an illustration of a variable stiffness snare device including a saline infusion port, in accordance with an embodiment of the present specification;

FIG. 11A is another illustration of a snare device comprising a variable size and stiffness level loop, in accordance with an embodiment of the present specification;

FIG. 11B shows the snare loop of the snare device of FIG. 11A being retracted to bring the snare loop in contact with saline solution infused within the sheath of the snare device; and

FIG. 12 is another illustration of a snare device comprising a plurality of valves, in accordance with an embodiment of the present specification.

### DETAILED DESCRIPTION

The present specification is directed towards an improved snare device which is configured such that the size of the snare loop can be reliably and predictably changed as per the requirement during a medical procedure, while maintaining the general shape of the snare loop. In embodiments, the present specification discloses a snare device which is configured such that the snare loop generally maintains its original shape as the size of the loop is modified within a defined range. In an embodiment, the present specification describes a snare device which is configured such that the size of the snare loop can be increased or decreased in discrete steps. In an embodiment, the present specification describes a snare loop which can assume up to three different sizes while maintaining the original shape of the loop.

In an embodiment, the present specification also describes a system and method to provide visual cues to a user indicative of the deployed size of the snare loop as the size is reduced or enhanced. In embodiments, the present specification also describes an improved snare device in which the stiffness level of the snare loop can be controlled for desirable effect.

“Treat,” “treatment,” and variations thereof refer to any reduction in the extent, frequency, or severity of one or more symptoms or signs associated with a condition.

“Duration” and variations thereof refer to the time course of a prescribed treatment, from initiation to conclusion, whether the treatment is concluded because the condition is resolved or the treatment is suspended for any reason. Over the duration of treatment, a plurality of treatment periods may be prescribed during which one or more prescribed stimuli are administered to the subject.

“Period” refers to the time over which a “dose” of stimulation is administered to a subject as part of the prescribed treatment plan.

The term “and/or” means one or all of the listed elements or a combination of any two or more of the listed elements.

The terms “comprises” and variations thereof do not have a limiting meaning where these terms appear in the description and claims.

Unless otherwise specified, “a,” “an,” “the,” “one or more,” and “at least one” are used interchangeably and mean one or more than one.

The term austenite finish (Af) temperature is a temperature above which a phase transformation from martensite to austenite is fully completed during heating of an alloy (such as Nitinol).

For any method disclosed herein that includes discrete steps, the steps may be conducted in any feasible order. And, as appropriate, any combination of two or more steps may be conducted simultaneously.

Also herein, the recitations of numerical ranges by endpoints include all numbers subsumed within that range (e.g., 1 to 5 includes 1, 1.5, 2, 2.75, 3, 3.80, 4, 5, etc.). Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present specification. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the specification are approximations, the numerical values set forth in the specific examples are reported as precisely as possible. All numerical values, however, inherently contain a range necessarily resulting from the standard deviation found in their respective testing measurements.

The present invention is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

It should be noted herein that any feature or component described in association with a specific embodiment may be used and implemented with any other embodiment unless clearly indicated otherwise.

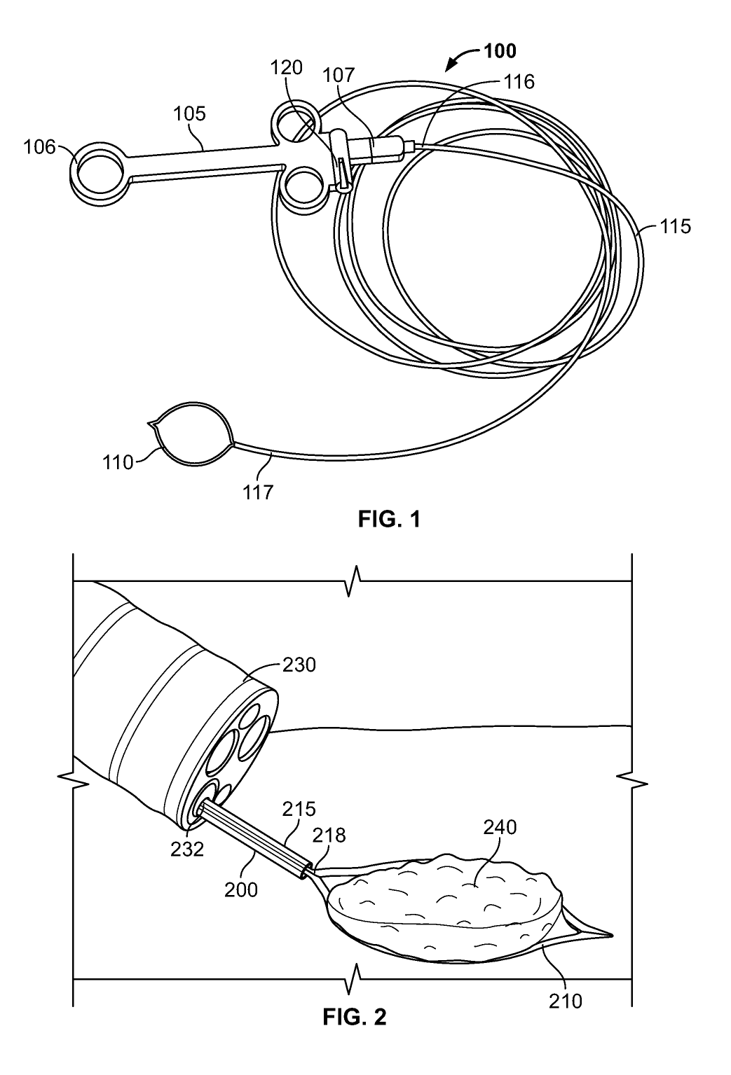

FIG. 1 is an illustration of a typical snare device 100, depicting a handle 105, snare loop 110, and a snare sheath 115. The handle 105 comprises a proximal end 106 and a distal end 107. The snare sheath 115 also comprises a proximal end 116 and a distal end 117 such that the proximal end 116 of the snare sheath is coupled to the distal end 107 of the handle 105 and the distal end 117 of the snare sheath allows for retraction and deployment of the snare loop 110. A snare wire is disposed within the snare sheath 115 such that a proximal end of the snare wire extends through the proximal end 116 of the sheath 115 and is connected to the handle 105 and a distal end of the snare wire extends through the distal end 117 of the snare sheath 115 and is connected to the snare loop 110. The snare sheath 115 and the wire present in the snare sheath 115 are controlled by the user through the handle 105. An operator holds the snare device 100 from its handle 105 and inserts the snare loop 110 and a portion of snare sheath 115 into the patient body through a medical device such as an endoscope. Once the snare loop 110 encircles the target tissue, such as a polyp, the loop 110 is withdrawn into the sheath 115 by pulling the wire present in the sheath 115 to sever the target tissue. The handle 105 comprises the means to control the length of snare sheath 115 which is lowered into the body. Further, the handle 105 comprises the means to extend or pull the wire present in the sheath 115 and hence manipulate the position and size of the snare loop 110 as per the requirement. If required, electrocautery can also be performed by applying a current to the wire loop 110 to assist in removing the polyp. The snare device 100 also includes a cautery or electrosurgical connector 120 for the same.

In the above embodiment, the medical practitioner can modify the size of the loop 110 by extending or pulling the wire present in the snare sheath 115. However, in practice, as the wire coupled to the loop 110 is extended or pulled, the shape of the loop 110 is distorted. For example, in case the wire present in the sheath 115 is pulled inwards (towards the handle), a portion of the loop 110 is withdrawn into the sheath 115 which reduces the size of loop 110, however the loop 110 becomes relatively narrow and elongated in shape. Similarly, in case the wire present in the sheath 115 is extended outwards (away from the handle), a size of the loop 110 expands however the loop 110 becomes relatively more round in shape.

In medical procedures, tumors or polyps might be of various sizes and in case the snare loop size is not aligned with the size of the polyp, the practitioner is required to completely withdraw the snare device and insert an alternate snare device, which makes the entire process very tedious. It is important to have snare devices in which the size of the snare or the wire loop can be modified without distorting the shape of the loop. A variable size snare loop simplifies the medical procedures by allowing a practitioner to resize the snare loop without completely removing the snare device and inserting an alternate snare device.

FIG. 2 is an illustration of a snare device 200 deployed in the working channel 232 of an endoscope 230, depicting a snare loop 210 encircling a target tissue 240. As shown in FIG. 2, the snare device 200 comprises a sheath 215. A wire 218 disposed in the sheath 215 emerges from its distal end and is connected with the snare loop 210. Typically, the diameter of the snare sheath 215 is such that it can easily be deployed in a working channel of commercially available endoscopes. A handle (not shown) coupled to the proximal end of the snare sheath 215 is used to manipulate the position and the movement of loop 210 through the wire 218. The handle also comprises the means to control the length of snare sheath 215 which is lowered into the body. In FIG. 2, as the snare loop 210 is encircling the target tissue 240, the target tissue 240 can be severed by withdrawing the loop 210 into the snare sheath 215 by pulling the wire 218 coupled to the loop 210. The snare loop 210 not only needs to be flexible to conform to the shape of the target tissue 240 but also needs to be stiff enough to encircle the tissue 240 and apply pressure on the surrounding tissue so as to grasp the encircled tissue 240 while the snare loop 210 is closed. If the loop 210 is too stiff, it will not properly encircle and conform to the tissue 240 to be resected and if the loop 210 is too soft it will slip over a flat tissue while the loop 240 is being closed. It is therefore desirable that the loop be soft while the snare loop is placed around the target tissue but then stiffen up to adequately grasp the tissue while the snare loop is closed.

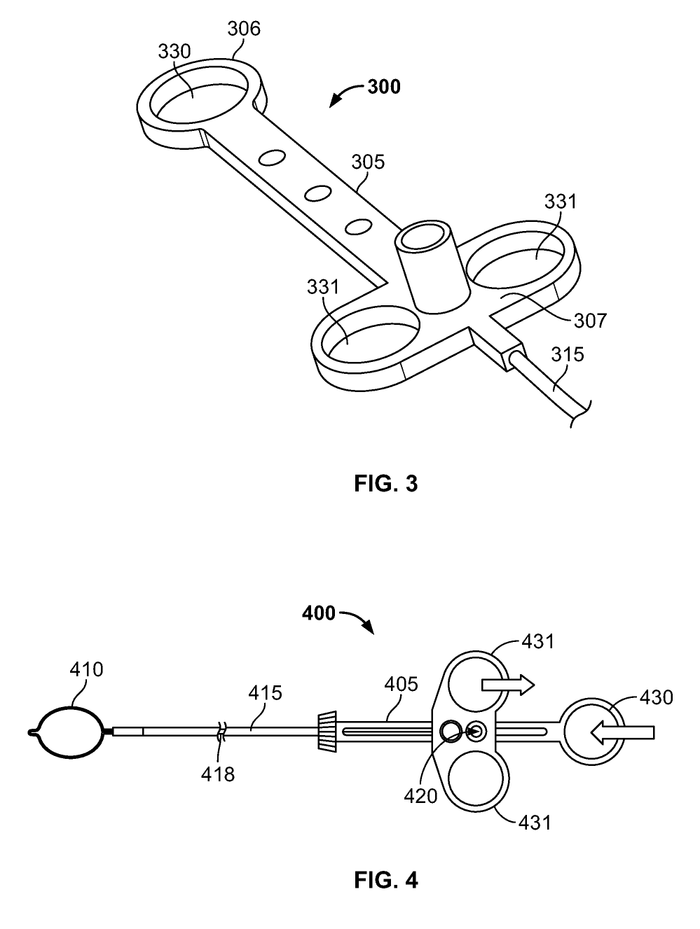

FIG. 3 is an illustration of a snare device handle 305 of a snare device 300. The handle 305 comprises a proximal section 306 and a distal section 307. The proximal section 305 comprises one finger hole 330 and the distal section 307 comprises two finger holes 331 for manipulating the snare device. The handle 305 is shown coupled to a snare sheath 315 which comprises a wire that emerges from a distal portion of the snare sheath 315 and is coupled to a snare loop as depicted in FIG. 1 and FIG. 2.

FIG. 4 is another illustration of a snare device 400, depicting a snare handle 405, snare loop 410, and snare sheath 415. The handle 405 is coupled to a snare sheath 415 that comprises a snare wire 418 inside it. The snare wire 418 emerges from the distal end of the snare sheath 415 and is coupled to the snare loop 410 at its distal end. The proximal end of the snare wire 418 is coupled to the snare handle 405 such that a user can control the snare wire 418 and therefore the snare loop 410 coupled to it through the handle 405. The handle 405 comprises a finger hole 430 at its proximal end and two other finger holes 431 located towards its distal end. In an embodiment, the snare device 400 is structured such that a user can insert his thumb in the finger hole 430 and one finger each (preferably the index finger and the middle finger) in the two finger holes 431. The user can then extend the snare wire 418 by pushing the finger holes 431 away from the finger hole 430 and can withdraw the snare wire 418 by pulling the finger holes 431 closer to the finger hole 430.

To sever a target tissue such as a polyp, once the snare loop 410 encircles the target tissue, the loop 410 is drawn into the snare sheath 415 by pulling the wire 418 coupled to the loop 410. In an embodiment, the snare wire is pulled by moving the finger holes 431 closer to the finger hole 430.

The handle 405 can also control the size of the snare loop 410 by extending or pulling the wire 418 coupled to the snare loop 410. However, modifying the size of the snare loop 410 distorts it shape. For example, in case the wire 418 present in the sheath 415 is pulled inwards (towards the handle 405), a portion of the loop 410 is withdrawn into the sheath 415 which reduces the size of loop 410, however the loop 410 becomes relatively narrow and elongated in shape. Similarly, in case the wire 418 present in the sheath 415 is extended outwards (away from the handle 405), the size of the loop 410 expands however the loop 410 becomes relatively more round in shape.

If required, electrocautery can also be performed by applying a current to the wire loop 410 to assist in removing the polyp. The snare device 400 includes a cautery or electrosurgical connector 420 for the same.

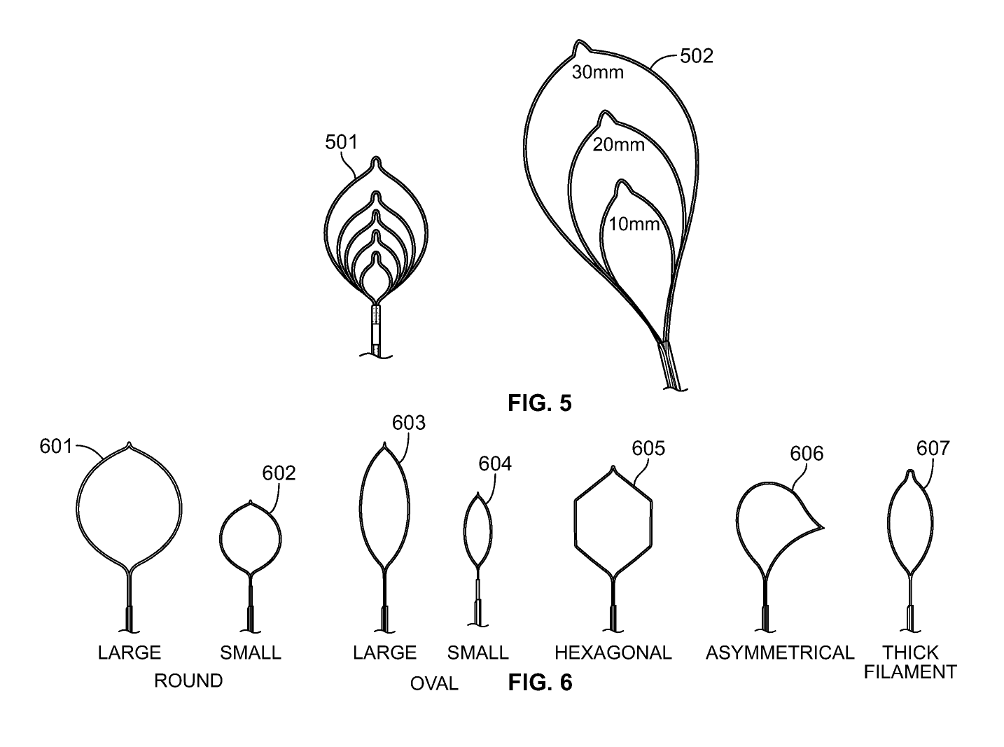

FIG. 5 is an illustration of multiple snare loops 501, 502, configured such that their respective loop size can be modified. Snare loop 501 can be configured to assume a variety of sizes as shown in FIG. 5. The size of the snare loop 502 can be changed among three different configurations such that it can assume a width of 10 mm or, 20 mm or 30 mm. One can notice that as the size of the snare loops 501 and 502 are reduced the shape of the snare loop is also distorted. In the images shows in FIG. 5, as the size of the snare loop 501 or 502 is reduced, the shape of the respective loop becomes relatively elongated and stretched as compared to a round shape.

FIG. 5 illustrates how the snare size is controlled in typical snare devices. From the above illustration, one can appreciate that there exists no good means of measuring the size of the snare loop and adjusting the size also distorts the shape of the loop. The size becomes more narrow and long while it is preferred to be round in shape.

A target tissue such as a polyp can have any type of shape such as a round shape or a completely asymmetrical shape. To operate on different types of such target tissues, snare loops are typically available in a variety of shapes. FIG. 6 is an illustration of a variety of snare loops having different shapes. As shown in FIG. 6, the snare loops include a large round shaped snare loop 601, small round shaped snare loop 602, large oval shaped snare loop 603, small oval shaped snare loop 604, hexagonal shaped snare loop 605, asymmetrical snare loop 606, and thick filament shaped snare loop 607. A healthcare facility may typically stock snare loops of various different shapes.

The present specification is directed towards an improved snare device which is configured such that the size of the snare loop can be reliably and predictably changed as per the requirement during the medical procedure. In an embodiment, the present specification discloses a snare device which is configured such that the snare loop generally maintains its original or desired shape as the size of the loop is modified. In an embodiment, the present specification also describes a system and method to provide visual cues to a user indicative of the varying size of the snare loop as the size is reduced or enhanced. In embodiments, the present specification also describes an improved snare device in which the stiffness level of the snare loop can be controlled for desirable effect.

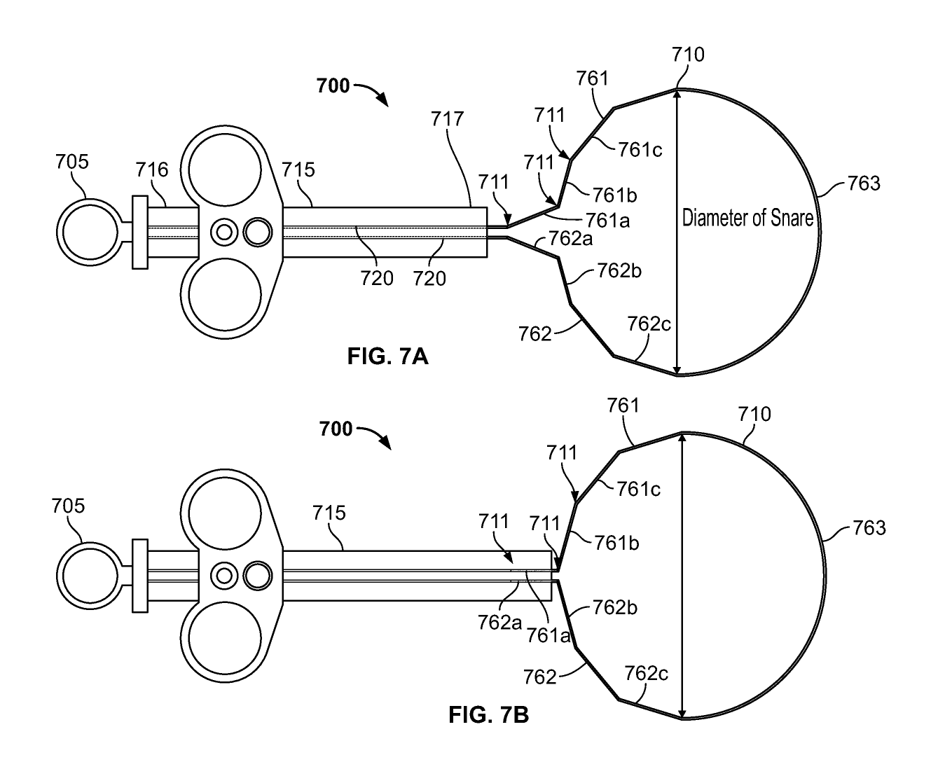

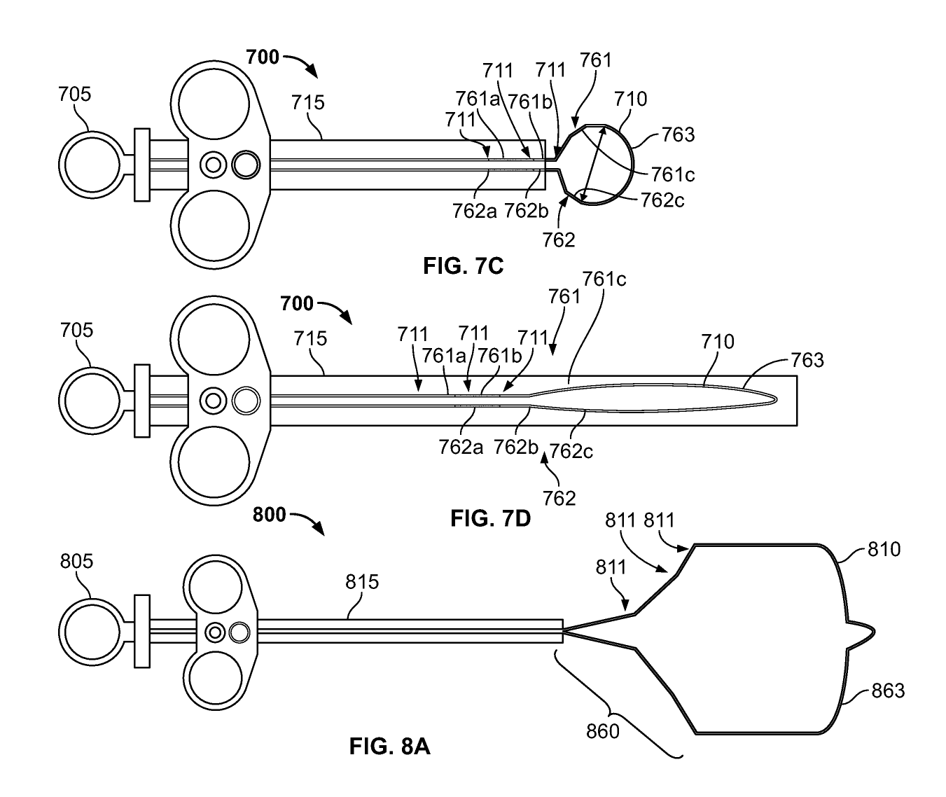

FIG. 7A illustrates a snare device in accordance with an embodiment of the present specification. As shown in FIG. 7A, the snare device 700 comprises a handle 705 coupled to a snare sheath 715. The snare sheath 715 has a proximal end 716 which is coupled to the handle 705 and a distal end 717 which is coupled to a snare loop 710. A wire, such as a pair of wires 720 shown in the embodiment of FIG. 7A, is disposed within the snare sheath 715 such that a proximal end of the wire 720 extends from the proximal end 716 of the sheath 715 and is connected to the handle 705 and a distal end of the snare wire 720 extends from the distal end 717 of the sheath 715 and is connected to the snare loop 710. The snare sheath 715 and the wire 720 present in the snare sheath 715 are controlled by an operator through the handle 705.

In an embodiment, the snare loop 710 comprises a first section 761 and a second section 762 wherein the distal ends of both first section 761 and the second section 762 are coupled to a third section 763. The proximal ends of first section 761 and second section 762 are coupled to the snare wire 720 that is disposed within the snare sheath 715. In an embodiment, the third section 763 comprises almost half of the total perimeter of the snare loop 710 and is substantially circular in shape. In an embodiment, the first section 761 further comprises a plurality of sub sections such as sub sections 761a, 761b and 761c and the second section 762 comprises a plurality of subsections such as sub sections 762a, 762b and 762c. In embodiments, each of subsections such as 761a, 761b, 761c, 762a, 762b and 76c is coupled to other sections of the snare loop at different angulations 711. In an embodiment, the length and angular orientation of subsection 761a is aligned to that of subsection 762a and the length and angular orientation of subsection 761b is aligned to that of subsection 762b and the length and angular orientation of subsection 761c is aligned to that of subsection 762c. In embodiments, the size of the snare loop 710 can be modified by extending or retreating portions of first section 761 and second section 762 in the sheath 715. In an embodiment, the size of the snare loop 710 can be modified in discrete steps to reduce the size of the snare loop 710 such that subsections 761a and 762a are simultaneously withdrawn inside the snare sheath 715. Similarly, subsections 761b and 762b can be simultaneously withdrawn inside the snare sheath 715 to further reduce the size of the snare loop 710. To even further reduce the size of the snare loop 710, subsections 761c and 762c are also withdrawn into the snare sheath 715. As the corresponding subsections, such as subsections 761a and 762a, are aligned with each other in terms of length and angular orientation, as the size of the snare loop 710 is modified in discrete steps, the snare loop 710 substantially maintains its original shape. In other words, the subsections 761a, 761b, 761c, 762a, 762b and 762c, due to their length and angular orientations, ratchet down the third section 763, as the size of the snare loop 710 is reduced in discrete steps, while still substantially maintaining the original shape of the loop 710.

In an embodiment, the snare loop 710 is manufactured from shape memory alloy such as Nitinol and has two different stiffness levels across certain ranges of temperature. In an embodiment, snare loop 710 has a first stiffness level below 30 degrees Celsius temperature and a second stiffness level above 30 degrees Celsius temperature, wherein the second stiffness is greater than the first stiffness.

FIG. 7B, FIG. 7C and FIG. 7D illustrate how the size of the snare loop is modified in discrete steps without distorting the shape of the snare loop, in accordance with an embodiment of the present specification. As shown in FIG. 7B, the subsections 761a and 762a have been together withdrawn into the snare sheath 715 such that the size of the snare loop 710 is reduced without distorting the original shape of the snare loop 710 as depicted in FIG. 7A. The presence of section 763 which, in an embodiment, is substantially circular or round in shape, ensures that as the subsections 761a and 761b are withdrawn into the snare sheath 715, the shape of the snare loop 710 is not distorted and it retains its original shape, substantially. The angulations 711 ensure that the portion of loop 710 which remains deployed retains its shape when a sub-portion of the loop 710 is drawn into the sheath 710. One can appreciate that in FIG. 7B, as the subsections 761a and 761b are withdrawn into the snare sheath 715, the snare loop 710 is not elongated or narrowed which is encountered with currently available snare loops when their size is modified. In FIG. 7C, both pairs of corresponding sections 761a, 762a and 761b, 762b are withdrawn into the snare sheath 715 such that the size of the snare loop 710 is further reduced while the original shape of the loop 710 is still substantially maintained. In FIG. 7D, the complete snare loop including all the three pairs of discrete segments 761a, 762a and 761b, 762b and 761c, 762c are withdrawn into the snare sheath 715.

In some embodiments, the present specification also describes a system and method to provide visual cues indicative of the size of the snare loop to a user. The angulations 711 or the specific subsections, such as the subsections 761a, 762a and 761b, 762b and 761c, 762c between the angulations 711, are colored to give the operator visual clue indicative of the size of the snare loop 710. In accordance with an aspect, the angulations 711 between the subsections, such as the subsections 761a and 761b or 761b and 761a or 762a and 762b or 762b and 762c, are less than 180 degrees. The wires, or subsections, in the inner section are bending inward creating a less than 180 degrees bend.

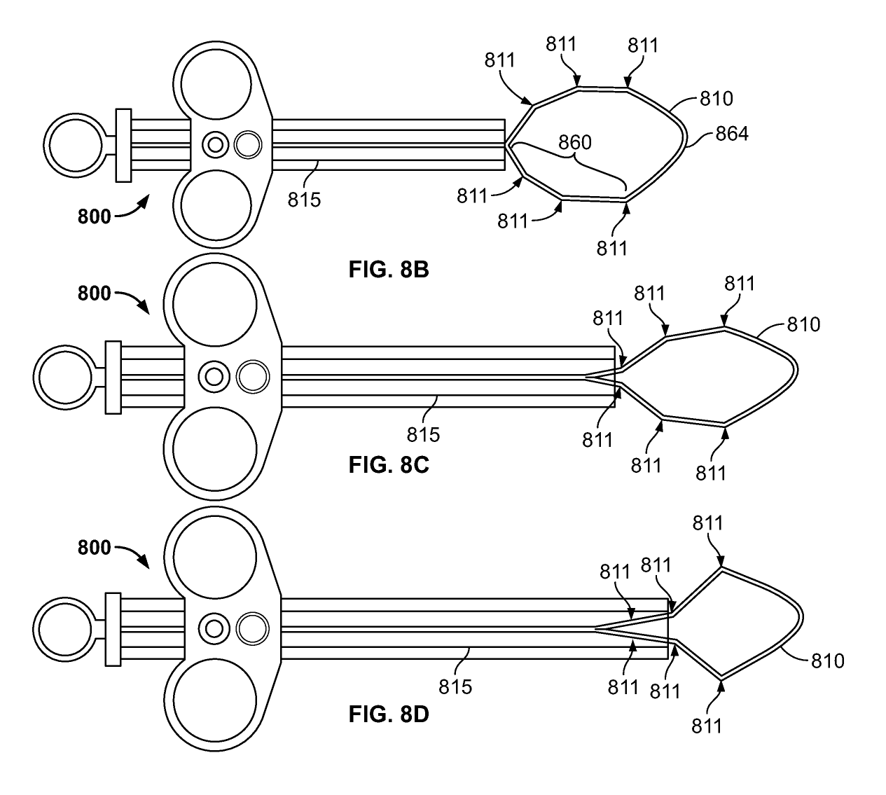

FIG. 8A is an illustration of a snare device 800 having discrete bends, angulations or angles 811 in the snare loop 810, in accordance with another embodiment of the present specification. The discrete bends, angulations or angles 811 in the loop 810 allow for constriction of the snare loop 810 in discrete segments. Optionally, the snare device 800 having discrete bends or angles 811 includes a snare loop 810 having a distal portion 863 which is predominantly square-shaped and a proximal portion 860 with angulations or discrete bends 811. FIG. 8B illustrates the snare device 800 having discrete bends or angulations 811 at the proximal portion 860 of the loop 810 while the distal portion 864 is round or circular or ovoid or elliptical shaped in accordance with some embodiments. In accordance with an aspect, the outer angulations or discrete bends 811, of the proximal portion 860 of the loop 810, are greater than 180 degrees. In accordance with various aspects, the distal portion 863 or 864 comprises approximately half of the loop 810 while the proximal portion 860 comprises the other approximate half of the loop 810. In various embodiments, however, the distal portion 863 or 864 may comprise more than 25% and up to 90% of the loop 810.

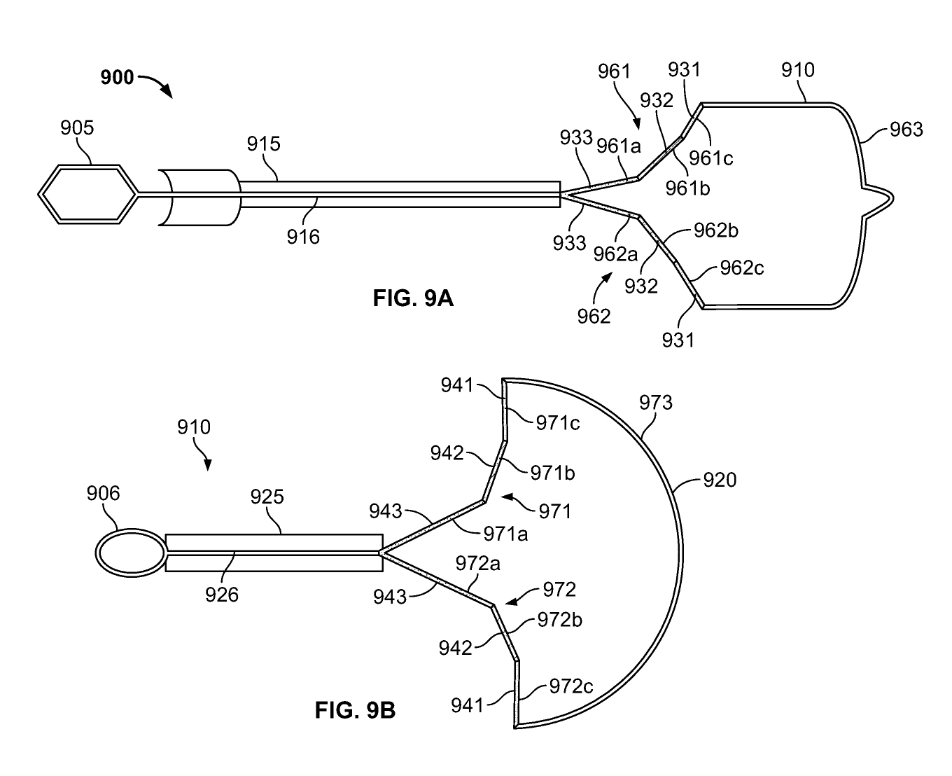

FIGS. 8C and 8D illustrate the snare loop 810 being withdrawn into the sheath 815 of the snare device 800, in accordance with another embodiment of the present specification. As the angulations 811 are constricted by closing the snare loop 810, the snare loop 810 substantially maintains its general original shape such as the round shape 864 of FIG. 8B (or the square shape 863 of FIG. 8A in another embodiment). In accordance with some embodiments, markings are provided at the angulations 811 or the segments between the angulations 811 are colored, as described further with reference to FIGS. 9A and 9B, to give the operator visual clue of the corresponding size of the snare loop 810.

It should be appreciated that the angulations or discrete bends 811 works as a ratchet mechanism ratcheting the loop 810 down while still maintaining a substantially general shape of the loop 810. In contrast, prior art snare loops turn oval as the loop compresses more along the breadth of the loop as compared to the length of the loop.

FIG. 9A illustrates a snare device 900 having different colors or markers on the snare loop 910 to indicate the size of the loop, in accordance with an embodiment of the present specification. As shown in FIG. 9A, the snare device 900 comprises a handle 905 coupled to a wire 916 within a snare sheath 915 which is in turn coupled to a snare loop 910. In an embodiment, the snare loop 910 comprises a first section 961, a second section 962, and a third section 963 such that the proximal ends of sections 961 and 962 are coupled to the wire 916 within the snare sheath 915 and the distal ends of the sections 961 and 962 are coupled to two separate ends of a third section 963. In an embodiment, the first section 961 further comprises a plurality of sub sections, such as sub sections 961a, 961b and 961c, and the second section 962 comprises a plurality of subsections, such as sub sections 962a, 962b and 962c. In embodiments, each of subsections, such as 961a, 961b and 761c, is coupled to other sections of the snare loop at specific angulations. In an embodiment, the length and angular orientation of subsection 961a is aligned to that of subsection 962a and the length and angular orientation of subsection 961b is aligned to that of subsection 962b. Further, the length and angular orientation of subsection 961c is aligned to that of subsection 962c. In an embodiment, the size of the snare loop can be modified in discrete steps to reduce the size of the snare loop such that subsections 961a and 962a are simultaneously drawn inside the snare sheath 915. Similarly, subsections 961b and 962b can be simultaneously withdrawn inside the snare sheath 915 to further reduce the size of the snare loop 710. To even further reduce the size of the snare loop 910, sub sections 961c and 962c are also simultaneously withdrawn into the snare sheath 915. To provide visual cues to a user indicating the portion or size of the loop that is deployed at any given moment, in an embodiment, the present specification describes marking the angulations or segments between the angulations in different colors. As shown in FIG. 9A, the subsections 961a and 962a are marked in a first color 933. Similarly the subsections 961b and 962b are marked in a second color 932, different from the first color 933, and the subsections 961c and 962c are marked in a third color 931, different from the first color 933 and second color 932. In FIG. 9A, color 933 is a red color, color 932 is a color and color 931 is a green color. In other embodiments, any other colors can be used to differentiate various segments or subsections between the angulations. Providing different colors or markers on the wire provides the operator a visual assessment of the deployed size of the loop without needing to check the actual measurements. For example, in an embodiment, the operator may observe, on a monitor providing an image of the snare device within a patient's body, the colors or markings to determine the deployed size of the snare loop. To reduce the size of the snare loop 910, the user pulls on handle 905, which is attached to a proximal end of wire 916, which sequentially pulls subsections 961a, 962a, 961b, 962b, 961c, 962c into the sheath 915. Alternatively, in other embodiments, there are markings on the snare handle for the operator to observe on a monitor to determine the deployed size of the snare loop.

FIG. 9B illustrates another snare device 910 having colors or markers on the snare loop 920 to indicate the size of the loop, in accordance with an embodiment of the present specification. The snare device depicted in FIG. 9B is similar to that depicted in FIG. 9A except that the shape of the various sections such as the sections 971, 972 and section 973 are different in FIG. 9B as compared to the shape of corresponding sections in FIG. 9A. The different colors or markers 941, 942, 943 on the loop wire 920 provide the operator a visual assessment of the deployed size of the loop without needing to check the actual measurements. Though having a different shape than the device depicted in FIG. 9A, the device of FIG. 9B operates in a similar fashion. To reduce the size of the snare loop 920, the user pulls on handle 906, which is attached to a proximal end of wire 926, which is attached at its distal end to subsections 971a, 972a, to sequentially pull subsections 971a, 972a, 971b, 972b, 971c, 972c into the sheath 925.

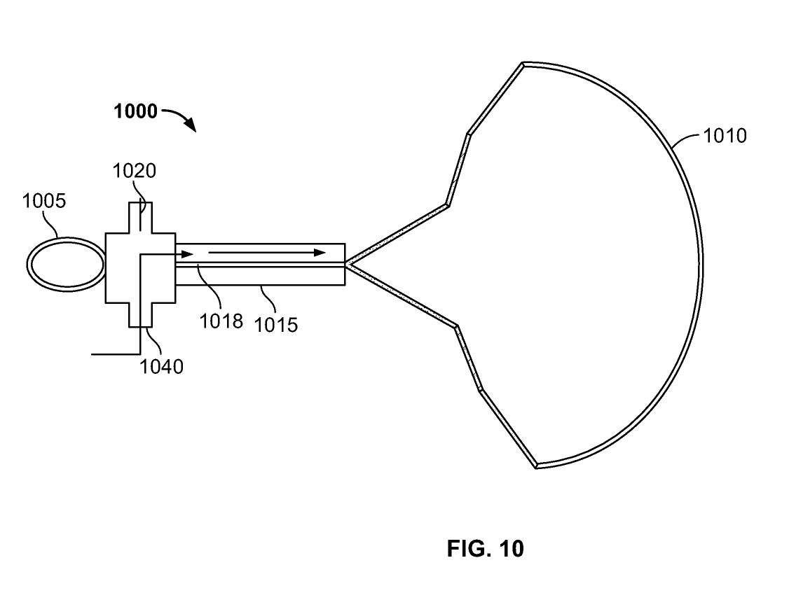

FIG. 10 is an illustration of a variable stiffness snare device 1000 including a saline infusion port 1040, in accordance with an embodiment of the present specification. As shown in FIG. 10, the snare device 1000 comprises a handle 1005 positioned at a proximal end a snare sheath 1015. At a distal end of the sheath 1015 is positioned a snare loop 1010. A wire 1018 is disposed within the snare sheath 1015 such that a proximal end of the wire extends from the proximal end of the sheath 1015 and is connected to the handle 1005 and a distal end of the snare wire extends from the distal end of the sheath 1015 and is connected to the snare loop 1010. The snare sheath 1015 and the wire 1018 present in the snare sheath 1015 are controlled by an operator through the handle 105.

In an embodiment, the snare loop 1010 is manufactured from a shape memory alloy, such as Nitinol, and has different stiffness levels across certain ranges of temperatures. In an embodiment, snare loop has a first stiffness level below 30 degrees Celsius temperature and a second stiffness level above 30 degrees Celsius temperature. In an embodiment, the handle 1005 comprises an infusion port 1040 such that a saline solution, having a thermal temperature less than the austenite finish (Af) temperature of the Nitinol, is injected in the snare sheath 1015 through the infusion port 1040. Contact with the saline solution infused through the port 1040 reduces the stiffness of the snare loop 1010 present inside the snare sheath 1015 that comes in contact with such saline solution. In the above embodiment, the stiffness of the snare loop 1010 when inside the sheath 1015 and in contact with saline at temperature <30° C. is lower than the stiffness of loop wire 1010 when it is pushed out of the sheath 1015 and is in contact with the tissue which has a temperature of 37° C. In an embodiment, the snare loop is pulled in and out of the sheath 1015 to vary the stiffness level of the deployed loop 1010 from a first stiffness when in the sheath to second, higher stiffness when outside the sheath. The snare device 1000 also includes an electrocautery port 1020.

It should be appreciated that the snare loop 1010 is desired to be soft, that is of the first stiffness, when being placed around a target tissue and needs to be stiffer, that is of the second stiffness, when the target tissue is constricted (similar to a noose). Thus, in accordance with an aspect of the present specification, the loop 1010 can be pulled in and out of the sheath 1015 to vary its stiffness between the first and the second stiffness levels.

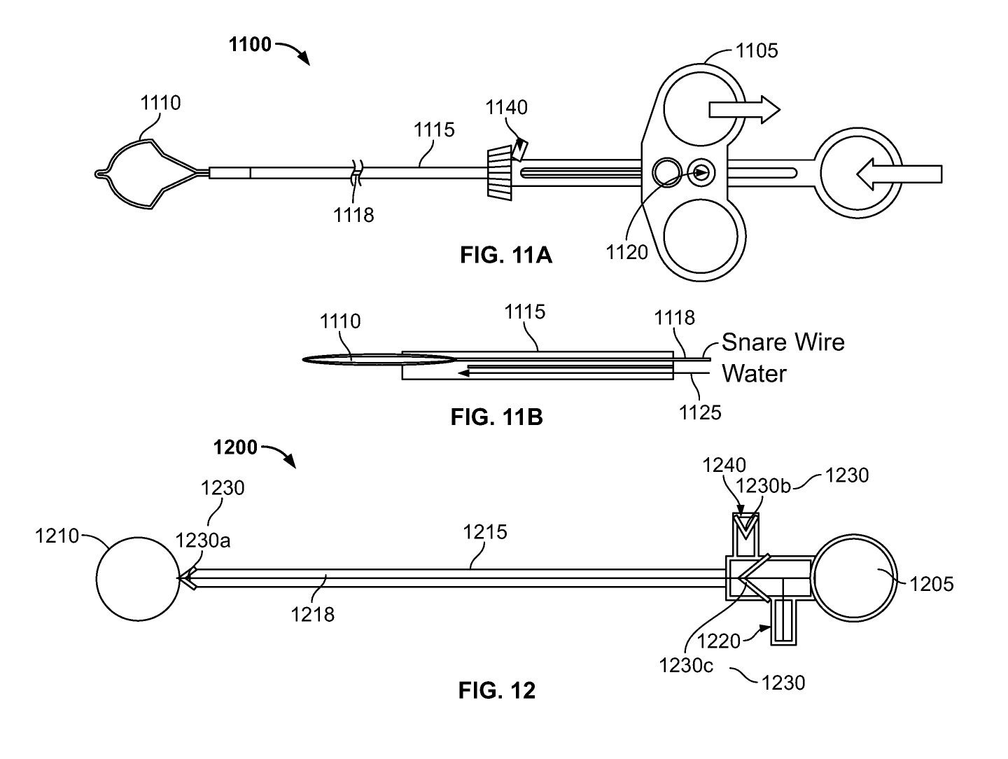

FIG. 11A is another illustration of a snare device comprising a variable size and stiffness level loop, in accordance with an embodiment of the present specification. In an embodiment, the snare device 1100 comprises a handle 1105 positioned at a proximal end of a snare sheath 1115. A variable size snare loop 1110 is positioned at a distal end of the sheath 1115. As shown in FIG. 11, the handle 1105 is coupled to a snare wire 1118 inside the sheath 1115. The snare wire 1118 emerges from the distal end of the snare sheath 1115 and is coupled to the snare loop 1110 at its distal end. The proximal end of the snare wire 1118 is coupled to the snare handle 1105 such that a user can control the snare wire 1118 and therefore the snare loop 1110 coupled to it through the handle 1105. In an embodiment, the snare loop 1110 and the wire 1118 is manufactured from a shape memory alloy, such as Nitinol, and has different stiffness levels across certain ranges of temperatures. The handle 1105 comprises an infusion port 1140 through which a saline solution or any other fluid at a suitable temperature can be infused to change the temperature of the wire 1118 present in the sheath 1115 and hence modify its stiffness level. The snare loop is pulled in and out of the sheath 1115 to vary the stiffness level of the deployed loop 1110. The snare device 1100 also includes an electrocautery port 1120. FIG. 11B shows the snare loop 1110 being retracted in the sheath 1115, by pulling the wire 1118, in order to bring the snare loop 1110 in contact with water or saline solution 1125 at a temperature less than Af temperature infused within the sheath 1115.

FIG. 12 is another illustration of a snare device 1200 comprising a variable size and stiffness level loop 1210, in accordance with another embodiment of the present specification. The snare device 1200 comprises a handle 1205 positioned at a proximal end of a snare sheath 1215. A variable size snare loop 1210 is positioned at a distal end of the sheath 1115. As shown in FIG. 12, the handle 1205 is coupled to a snare wire 1218 inside the sheath 1215. The snare wire 1218 emerges from the distal end of the snare sheath 1215 and is coupled to the snare loop 1210 at its distal end. The proximal end of the snare wire 1218 is coupled to the snare handle 1205 such that a user can control the snare wire 1218 and therefore the snare loop 1210 coupled to it through the handle 1205. The handle 1205 comprises an infusion port 1240 through which a saline solution or any other fluid at a temperature less than Af temperature can be infused to change the temperature of the loop 1210 present in the sheath 1215 and hence modify its stiffness level. The snare device 1200 also includes an electrocautery port 1220. In accordance with an aspect, the snare device 1200 further comprises a plurality of one-way valves 1230 to allow water or saline solution to flow in one direction only and not leak out of the snare device 1200. In one embodiment, the plurality of valves 1230 comprise a first valve 1230a positioned at the distal end of the sheath 1215, a second valve 1230b positioned at the infusion port 1240 and a third valve 1230c positioned at the electrocautery port 1220. The plurality of valves 1230 enable a saline solution or water column to be maintained at all times within the sheath 1215, in contact with the snare wire 1218, thereby obviating a need to constantly flush water within the sheath 1215. Additionally, when electrocautery is being performed it is desirable for the saline solution or water to not escape or leak out of the snare handle 1205 and electrocute the user. The intended valve function can be accomplished by valves known in the field including O-rings to prevent leakage or back flow of water and can be accomplished with one or more valves.

The above examples are merely illustrative of the many applications of the system of the present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A variable size snare device comprising:
a snare sheath comprising a proximal end and a distal end;
a snare wire comprising a proximal end and a distal end and disposed within said snare sheath;
a handle coupled to the proximal end of the snare wire; and
a snare loop coupled to the distal end of the snare wire, said snare loop comprising:
a first section having a proximal end and a distal end, the proximal end coupled to the distal end of the snare wire;
a second section having a proximal end and a distal end, the proximal end coupled to the distal end of the snare wire; and
a third section coupled to and between the distal end of first section and the distal end of second section;
wherein, each of said first section and second section comprises a plurality of angulations such that the loop sections between such angulations in the first section are aligned with the corresponding loop sections between such angulations in the second section.

2. The variable size snare device of claim 1, wherein the number of angulations and the degree of angulations in the first section is similar to that in the corresponding angulations in the second section.

3. The variable size snare device of claim 1, wherein each of said first section and said second section comprise three angulations.

4. The variable size snare device of claim 1, wherein the length of loop section between any two angulations in the first section is equal to the length of loop section between corresponding set of angulations in the second section.

5. The variable size snare device of claim 1, wherein the size of the snare loop is increased or decreased by extending or pulling portions of snare loop inside the sheath without substantially distorting the original shape of the snare loop.

6. The variable size snare device of claim 1, wherein the presence of said angulations ensure that the general shape of the snare loop is not substantially distorted as portions of snare loop are drawn into the snare sheath.

7. The variable size snare device of claim 1, wherein the size of the snare loop is increased or decreased in discrete steps without substantially distorting the original shape of the snare loop by extending or withdrawing the entire loop sections between any two angulations in the snare sheath.

8. The variable size snare device of claim 1, wherein said third section is substantially circular in shape.

9. The variable size snare device of claim 1, wherein the loop sections between any two angulations are colored in different colors to provide a visual cue on a monitor indicative of the size of deployment of the snare loop to an operator.

10. The variable size snare device of claim 1, wherein the angulations are marked in different colors to provide a visual cue indicative of the size of deployment of the snare loop to an operator.

11. The variable size snare device of claim 1, wherein said snare wire disposed inside said snare sheath comprises two wires such that the proximal ends of the wires are coupled to the handle and the distal ends of the wires are coupled to the snare loop and the position and size of the snare loop is controlled through said wires.

12. The variable size snare device of claim 11, wherein said snare loop is manufactured from a shape memory alloy and has different stiffness levels across certain range of temperature.

13. The variable size snare device of claim 12, wherein said handle comprises an infusion port to infuse a suitable fluid into the snare sheath to modify the temperature and stiffness level of the snare loop.

14. The variable size snare device of claim 12, wherein said shape memory alloy comprises Nitinol.

15. The variable size snare device of claim 14, wherein said handle comprises an infusion port to infuse a saline solution having a thermal temperature less than the austenite finish (Af) temperature of the Nitinol.

16. The variable size snare device of claim 1 further comprising an electrosurgical connector to facilitate electrocautery.

17. A variable size snare device comprising:
a snare sheath; and
a snare wire positioned within said sheath and attached to a handle at one end and having a snare loop at the other end, wherein the snare loop includes a plurality of angulations which allow the snare loop to substantially maintain its shape as it is withdrawn into said snare sheath.

18. A snare device with variable stiffness level comprising:
a snare sheath;
a handle coupled to the proximal end of the snare sheath and comprising an infusion port to infuse fluid inside said sheath; and
a snare wire positioned within said sheath and attached to a handle at one end and comprising a snare loop at the other end, wherein the stiffness level of snare loop inside the snare sheath is modified as compared to the stiffness level of the snare loop outside by bringing it in contact with a fluid infused through said infusion port.

19. The snare device of claim 18, wherein the temperature of said fluid is set such that it affects the stiffness level of said snare loop.

20. The snare device of claim 18, wherein said snare loop is pulled in and out of the distal end of said sheath to change the size and overall stiffness level of deployed snare loop.
